(19) United States
(12) Patent Application Publication
(10) Pub. No.: US 2008/0319678 A1
(43) Pub. Date: Dec. 25, 2008

(54) Mass Tagging for Quantitative Analysis of Biomolecules using 13C Labeled Phenylisocyanate

(75) Inventors: Dennis J. Templeton, Earlysville, VA (US); Charles E. Lyons, JR., Palmyra, VA (US); Sergey Moshnikov, Charlottesville, VA (US); Janet V. Cross, Ruckersville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/279,076

(22) PCT Filed: Feb. 15, 2007
(86) PCT No.: PCT/US2007/004164

§ 371 Date: Aug. 12, 2008

Related U.S. Application Data
(60) Provisional application No. 60/773,446, filed on Feb. 15, 2006.

(51) Int. Cl. G01N 33/00; C12Q 1/02; G06F 19/00

(52) U.S. Cl. 702/19; 436/86; 435/29

(57) ABSTRACT

The present invention provides novel compositions and methods for mass-tagging peptides and proteins which are useful for identifying and quantifying mixtures of peptides and proteins.

### CROSS REFERENCE TO RELATED APPLICATIONS

This application claims priority under 35 U.S.C. § 119(e) to U.S. Provisional Application Ser. No. 60/773,446 filed Feb. 15, 2006, the disclosure of which is incorporated by reference in its entirety herein.

### BACKGROUND OF THE INVENTION

Mass spectrometric methods for quantifying proteins in complex mixtures is difficult. The most fruitful approaches rely on the use of chemical tags that impart a unique mass to peptides in the complex mixture, however currently available tags have definite restrictions in their utility.

Stable isotope mass tagging is of potential utility for quantitative or qualitative analysis of proteins present in complex mixtures, such as for the discovery of diagnostic biomarkers. However, practical application of this approach to biomarker discovery has been problematic because of difficulty in quantifying proteins in complex mixtures. One valuable approach is through the application of internally standardized protein mixtures through the application of stable isotope labeled mass tags, though currently available mass tags have significant limitations. These limitations include: the inability to label all peptides, complex labeling patterns that are difficult to interpret, changes in hydrophobic properties of peptides labeled with different isotopes, and difficulty in automated quantification.

There is a long felt need in the art for compositions and methods useful for mass tagging proteins to aid in quantitative and qualitative analysis of peptides and proteins, particularly mixtures of peptides and proteins. The present invention satisfies these needs.

### BRIEF SUMMARY OF THE INVENTION

The present invention provides a new stable ¹³C-labeled reagent, ¹³C phenylisocyanate (PIC), which together with conventional ¹²C PIC, serves as a mass-tagging reagent that labels peptides specifically at their amino termini and that offers significant advantages over other currently available mass tags.

The method of the Present invention using samples comprising complex mixtures of proteins labeled with ¹³C- and ¹²C-PIC provides much less standard variation in their measured abundance, far less than most current techniques. The present invention further provides analytical procedures that take advantage of unique features of the PIC label to enable rapid exclusion of unlabeled peptides that otherwise confounds marker discovery, and in addition assists in identification of amino-terminal b-ions, which assists in peptide identification. Application of this approach to complex clinical samples (nipple aspirates of breast cancer affected or contralateral breast) showed that the system allows rapid identification of peptides that are expressed at varying levels between specimens, and that therefore represent potential disease biomarkers. In one aspect, useful samples of the present invention include, but are not limited to, normal tissue samples, diseased tissue samples, biopsies, blood, saliva, feces, cerebrospinal fluid, semen, tears, and urine.

In one embodiment, the invention provides compositions and methods for producing labeled peptides that have increased charge in mass spectrometry. In one aspect, the invention provides ¹²C- and ¹³C-isocyanate labeling reagents comprising reversibly blocked amino groups. In one aspect, the reversibly blocked groups can be prepared using amino blocking reagents. In one aspect, the amino blocking reagents include, but are not limited to, BOC and FMOC. These methods provide chemically labeled peptides, that differ in mass by specific molecular weights, which have been deprotected using standard methods, resulting in mass tagged peptides with restored amino groups at the labeled amino terminus.

The invention further provides software (PICquant) that automatically and accurately identifies for each labeled peptide: charge state, root peptide mass, and abundance ratio between two samples. This new software enables an unconventional approach toward peptide marker identification, one that relies on detecting quantitative differences in PIC labeled peptides without the difficult and low-efficiency step of peptide sequence identification through database searching.

In one embodiment, PIC labeling is useful to improve the likelihood of automated database search algorithms through acquisition of improved determinations of peptide mass and through identification of b-ion and y-ions in MS2 fragmentation scans. This is possible because PIC labeling allows unequivocal identification of peptide fragments derived from the labeled amino terminus.

The present invention encompasses spectra are identified following analysis of the samples. In one aspect, the spectra are identified using a database search algorithm. In on aspect, the database search algorithm is selected from the group consisting of SEQUEST, MASSCOT, and OMSSA.

In one embodiment, the analysis determines peptide mass.

In another embodiment, the PIC label also enhances identification of sites of post-translational modification of proteins, by enabling comparison of spectra from modified or unmodified proteins without the necessity of sequence identification, which is difficult when searching for unknown modification types.

The present invention provides compositions and methods encompassing a complete system for peptide labeling, and manual and automated quantification and peptide identification that will be useful for many health-related discovery applications.

In one embodiment, the present invention provides a non-radioactive ¹³C-labeled chemical (¹³C-(6)-Phenylisocyanate, “PIC”). In one aspect, PIC can be used for specific labeling of amino termini of proteins and peptides.

In one embodiment, ¹²C-PIC and ¹³C-PIC, or substantially similar labels can be used to differentially modify proteins, peptides, or peptide mixtures in order to alter the mass of the peptides, proteins, or other biomolecules with minimal alterations of their chemical properties

In another embodiment, ¹²C- and ¹³C-PIC labels are useful for labeling proteins or peptides in analytical techniques in which molecule mass is determined, for example but not exclusively, MALDI mass spectroscopy and liquid chromatography/mass spectroscopy, gas/chromatography/mass spectroscopy, and tandem mass spectroscopy (LC/MS/MS).

In one embodiment, manual and computer assisted algorithms are provided to identify peptides and other molecules labeled with PIC or similar labels based on characteristic chemical reactions that occur during analysis.

In another embodiment the invention provides methods for optimized data acquisition schema during mass spectroscopy to maximize quantitative information useful for PIC-based quantification during LC/MS/MS or other mass-based analysis. In one aspect, manual and computer assisted algorithms are used to compare and quantify peptides in complex mixtures following mass analysis using PIC or similar mass labels by comparison of ion peaks representing proteins differentially labeled with ¹²C- or ¹³C-PIC labels. In another aspect, manual and computer assisted algorithms are used to compare and quantify peptides following mass analysis using PIC or similar mass labels through use of a “quantify first then identify” strategy. In yet another aspect, manual and computer assisted algorithms to enhance identification of proteins from which peptides derive based on identification of N-terminal b-ions or C-terminal y-ions, or similar strategies that are made possible through use of the amino-terminal PIC label. In a further aspect, manual and computer assisted analyses of protein modifications such as proteolysis, phosphorylation, or other post-translational analysis through use of PIC or substantially similar mass labels.

The present invention further provides tables useful for identifying fragmentation ions that are characteristic of specific PIC-modified amino acids. The present invention further provides for preparing such tables.

In one aspect, the invention provides compositions and methods useful for distinguishing ¹³C-PIC labeled peptides from ¹²C-PIC labeled peptides, or from peptides with other labels, and the method identifies the charge of a peptide ion labeled with ¹²C-PIC or ¹³C-PIC. In one aspect, samples can be labeled, but are combined before analysis. Comparison of the results of the analyses can be performed using manual algorithms or computer assisted algorithms to compare peptides.

One of ordinary skill in the art will appreciate that samples can be subjected to various treatments before analysis. For example, samples can be digested with enzymes before or after labeling. Trypsin is such an enzyme.

The present invention provides compositions and methods for mass-tagging and analyzing peptides and proteins obtained from various biological samples, which generally comprise complex mixtures of peptides. In one aspect, the invention provides methods for identifying and quantifying biomarker peptides and proteins indicative of various diseases, disorders, and conditions. In one aspect, the disease is cancer. In one aspect, the cancer is breast cancer.

In one aspect, the methods of the present invention are useful for identifying post-translational modifications of peptides or just the use of ¹²C-PIC or ¹³C-PIC as labels.

In one aspect, the present invention provides compositions and methods useful for validating that a peptide labeled with ¹³C-PIC comprises a ¹³C-PIC moiety. In another aspect, the present invention provides compositions and methods useful for validating that a peptide labeled with ¹²C-PIC comprises a ¹²C-PIC moiety.

The levels of labeled peptides can be quantified. The levels of unlabeled peptides can also be determined.

One of ordinary skill in the art will appreciate that the invention further encompasses methods for analyzing compounds other than peptides. Such compounds include, but are not limited to, nucleic acids, carbohydrates, and lipids. In one embodiment, the invention provides a method for identifying at least one compound in a sample, comprising obtaining a first sample; contacting an aliquot of said first sample with a first isocyanate moiety, or optionally a moiety comprising an isotope variant of a naturally occurring element, thereby labeling at least one compound with a first isocyanate moiety or a moiety comprising an isotope variant of a naturally occurring element in said aliquot; analyzing said aliquot to validate said first isocyanate moiety-labeling or said naturally occurring element-labeling of at least one compound; obtaining a second sample, wherein said second sample comprises an otherwise identical sample to said first sample or a second aliquot of said first sample, and contacting said second sample with a second isocyanate moiety; thereby labeling at least one compound with a second isocyanate moiety in said second sample; analyzing said second sample to validate said second isocyanate moiety-labeling of at least one compound; comparing the results of the analysis of said first sample with the results of the analysis of said second sample, thereby identifying at least one compound in a sample. In one aspect, a third sample can be obtained and labeled with a third label, and then compared to the first two samples.

In one aspect, the compound is a peptide. In one aspect, the method further identifies disease or disorder associated alterations in peptide structure. In another aspect, the invention further provides a method wherein said moiety comprising an isotope variant of a naturally occurring element comprises, for example, deuterium, ¹³C, ¹⁵N, and ¹⁸O. In yet another aspect, the invention provides compositions and methods wherein said moiety comprising an isotope variant of a naturally occurring element comprises protected amino groups that are subsequently chemically reversed.

In one embodiment, the present invention encompasses compositions and methods useful for identifying biomarkers characteristic of diseases and disorders. The method comprises obtaining a first sample from a subject with a disease or disorder and analyzing the sample according to the methods of the invention. In addition, to the first sample, the method further comprises obtaining a second otherwise identical sample from an unaffected subject and analyzing the second sample according to the methods of the invention. Then the results of the analysis of the first sample are compared with the results of the analysis of the second sample. A higher or lower level of a peptide in the first sample compared to the level of the peptide in the otherwise identical sample, is an indication that the peptide is a biomarker of a disease or disorder. In one aspect, the biomarker is a disease marker for cancer. In one aspect, the cancer is breast cancer. Samples obtained from subjects with a disease or disorder of interest include, but are not limited to, diseased tissue samples, biopsies, cultured cells, blood, saliva, feces, cerebrospinal fluid, semen, tears, and urine.

The present invention further provides compositions and methods useful for diagnosing a disease or disorder associated with a biomarker identified by the methods of the invention. The method encompasses obtaining a sample from a test subject, comparing the level of the biomarker of interest in the test subject with the level of the biomarker from an otherwise identical sample from an unaffected subject or from an otherwise identical unaffected sample from said test subject. A higher or lower level of the biomarker in the test subject, compared with the level of the biomarker in the sample from an unaffected subject, or from a standard sample or from an unaffected sample from the test subject, is an indication that the test subject has a disease or disorder associated with the biomarker.

### BRIEF DESCRIPTION OF THE DRAWINGS

FIG. 1. PIC labeling efficiency of six tryptic peptides derived from cerebrospinal fluid. The complex peptide mixture was labeled with (¹²C) PIC as described in Methods, using 1 mM or 2 mM final PIC concentrations for 15 minutes, as indicated. Both complex mixtures were analyzed using LC/MS/MS, and the resulting spectra analyzed using the SEQUEST algorithm against human proteins by specifying optional PIC labeling (119 Da) at amino termini and lysine residues. Peptides identified in both samples, modified or not with PIC, were quantified by centroid peak height from MS1 spectra. The table presents the percent of detectable peptide forms that were not labeled at all, or that were labeled at the amino terminus only or at both the amino terminus and the internal or CT lysine residues (no peptides were identified that labeled lysine only).

FIG. 2. Six ion chromatogram profiles of matched ion pairs labeled with ¹²C and ¹³C PIC. Labels indicate measured m/z and charge state. Observed elution times of three separate ion pairs show precise co-elution of all three pairs over a 60 minute LC gradient, including ions with charge of +1 (top 4 chromatograms) or +3 (bottom two chromatograms).

FIG. 3. Exact coelution of differently PIC-labeled peptides demonstrated by similar intensity ratios independent of location in elution profile. A complex protein mixture (unfractionated human CSF) was trypsinized and treated with either PIC-H or PIC-L, then the two otherwise identical samples mixed and analyzed in a single LC/MS/MS run. An abundant +1 ion was identified repetitively in both C¹³ and C¹² labeled samples, with a mass of 360 or 366 respectively, owing to repeated selection for MS2 analysis across the chromatographic elution profile that extended almost five minutes. High resolution (“zoom”) spectra as described in later figures were analyzed to quantify both 360 and 366 m/z ions, at the 17 times indicated above the elution chromatogram. Intensity ratios were measured at each of these points, and ¹²C/¹³C label ratios calculated at each point, as shown for each point. This ratio, theoretically 1.0, was nearly identical at all time points, and importantly did not change across the 4 minute time flame during which the samples were measured, and across a ten-fold variation in total ion intensity. Overall, the 17 determinations resulted in an average log₂ ratio of 1.02±0.80. Average log₂ ratios of the first eight determinations was 0.99±0.06 and of he last 9 determinations was 1.06±0.08, suggesting less than 10% variation across the elution peak.

FIG. 4. High resolution zoom scans enhance quantification of ion pairs. Several scans from analysis of the complex CSF sample described in FIG. 3 are presented, indicating ions that should be of identical intensity. In Panel A, a segment from a wide-range MS1 scan is shown in which several PIC-L/PIC-H ion pairs are evident, 4 of which are marked by brackets. Despite good concordance of these ion pairs, it is evident that imprecise quantification could result by splitting one ion peak into separate bins. Panels B-D show high resolution scans from this sample, in which isotope envelope peaks are clearly distinguished. Panel B represents one of the +1 ion pairs in Panel A, while +2 and +3 ions are shown in Panels C and D. Particularly in spectra of lower intensity ions, the zoom scans result in more precise quantification, largely because of reduced ambiguity of which ion species are to be quantified.

FIG. 5. Forty-one peptide pairs were quantified from the CSF sample (containing identical proteins labeled with PIC-L or PIC-H) that were separately identified using SEQUEST specifying either ¹²C or ¹³C PIC labeling. Heavy and light ion species were quantified and ¹²C/¹³C labeling ratios were derived from wide-spectrum MS1 analysis (left column) or narrow range (“zoom”) scans (right column).

While average peak intensity derived both quantitative approaches are close to the theoretical ratio of 1.0, the zoom scan quantification almost always produces a nearly even ratio. The error resulting from analysis of MS1 intensities appears to result from confusion of sequenced peptides with nearby abundant ions that are poorly resolved in the wide-range scans. In biomarker discovery experiments, these incorrect measurements could be erroneously interpreted as potential biomarker candidates.

FIG. 6. Alternate approaches to quantification of high-complexity LC/MS/MS datasets. Most previous approaches have followed the approach on the left part of the schematic, to first identify peptides corresponding to spectra through database searching, and then to quantify the abundance of identified peptides. The PIC labeling strategy increases the facility of the approach on the right, because it simplifies quantification of spectral intensities on zoom scans, and because it improves identification of spectra of interest.

FIG. 7. Schematic overview of protocol to identify markers diagnostic of breast cancer. Under an IRB approved protocol, women diagnosed with breast cancer by core biopsy underwent ductal lavage of both the affected and contralateral breast. Protein rich samples were obtained that contain proteins present within breast ducts, that in principle will contain proteins released into the duct from intact or dying cancer cells. Samples were processed without knowledge of which breast in which the carcinoma arose.

FIG. 8. PIC labeled peptides produce characteristic ion fragments during MS2 analysis. Scrutiny of spectra from ductal lavage samples revealed the potential problem that peptides that failed to be labeled with either PIC-L or PIC H would have no mass-tagged partner ion, and would thus resemble singlet ions that could be incorrectly interpreted as potential biomarker proteins Panel A shows a zoom scan of a bona fide partner pair; the lowest m/z of the PIC-L labeled peptide was automatically selected by the spectrophotometer for MS2 analysis, shown in Panel B. These spectra are displayed with the m/z value of the selected ion set to the relative value of 0.0, so that ions detected in the MS2 spectra are specified with values below (or in the case of +2 or greater charge ions, above) the m/z of the selected ion. Characteristic neutral loss ions were observed that represented complete loss of the PIC label (PIC loss) or loss of the phenylamine moiety, comprising most of the label, but retention of an isocyanate moiety by the peptide (PhA loss). Representative MS2 fragments of PIC-labeled peptides are shown in panels B-E with ions derived from these neutral losses indicated by boxes or ovals. MS1 spectra are not shown that correspond to Panes C-E. Panel F shows a table with predicted neutral losses for peptides of various charges labeled with either PIC-L or PIC H

FIG. 9. Characterization of PIC-labeled peptides that were differentially expressed in ductal lavage samples. Of 15,000 pairs of zoom and MS2 spectra obtained in the first experiment, 80 peptide ions were identified using manual spectrum analysis that were both PIC labeled and were found at more than a 2-fold variance with their predicted ion partner. Data files (.dta) were collected for these 80 MS2 spectra and were compared with the human protein dataset using SEQUEST, through which peptide sequence identifications were made with an XCorr score greater than 1. Two of these peptide ID's indicated two different peptides of the human Polymeric Immunoglobin Receptor (PIgR). Subsequently, a fresh aliquot of ductal lavage proteins were trypsinized and PIC-labeled (reversing the PLC-H and PIC-L labels compared with Experiment 1). SEQUEST database searching revealed three peptides derived from PIgR (one not detected in Experiment 1 initially).

Manual analysis of ion elution profiles eventually identified MS2 spectra and zoom MS1 spectra for all four peptides in both samples. Representative zoom spectra (Panels A, C, E, and G) as well as corresponding MS2 spectra (Panels B, D, F, and H) are shown. As in FIG. 7, ion m/z values are compared relative to the m/z of the ion targeted for analysis, and the characteristic neutral loss in MS2 spectra are indicated. Quantification of PIC-H and PIC-L ion pairs was performed by comparing the dissimilar ion pairs (arrows in zoom MS1 scans) and ratios of all 8 determinations of PIgR-derived peptides are shown in Panel I, including both independent experiments. Overall, PIgR was identified in the cancer-affected breast at levels about 5-fold greater than in the control breast.

FIG. 10. Calculated masses of single and di-amino acids alone or conjugated with ¹²C-PIC or ¹³C-PIC. A. Mass of single amino acids are shown in the left column (with cysteine carboxymethylated using iodoacetamide) and the corresponding mass of the m+H]+b-ion calculated in the second column. The third and forth columns are the predicted mass of b-ions labeled with ¹²C-PIC or ³C-PIC respectively. B. A conventional dipeptide mass table, showing masses of combinations of two amino acids. C and D, the same table as in Panel B, but with one amino acid modified with ¹²C-PIC (Panel C) or ¹³C-PIC (Panel D). One or more of these tables are useful for identifying fragmentation ions that are characteristic of specific PIC-modified amino acids. For example, in FIG. 7D, a spectrum displaying fragmentation ions of a peptide with sequence IIEGPLNK, the ¹²C PIC-I monopeptide (233 m/z, labeled −333.18 relative to the analyte) and the ¹²C-PIC-II dipeptide (346 m/z, labeled −220.18 relative to the analyte) are clearly seen. In most other spectra, these labeled b-ions are evident as smaller peaks.

### DETAILED DESCRIPTION OF THE INVENTION

Abbreviations and Acronyms

CSF—means cerebrospinal fluid

PIC—means phenylisocyanate

¹²C PIC—means ¹²C phenylisocyanate

¹³C PIC—means ¹³C phenylisocyanate, also referred to as ¹³C-(6)-phenylisocyanate

PIC-H—is an alternate abbreviation for ¹³C-(6)-phenylisocyanate

PIC-L—is an alternate abbreviation for ¹²C phenylisocyanate

PIgR—means polymeric immunoglobin receptor

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

A disease, disorder, or condition is “alleviated” if the severity of a symptom of the disease or disorder, the frequency with which such a symptom is experienced by a patient, or both, are reduced.

The term “alterations in peptide structure” as used herein refers to changes including, but not limited to, changes in sequence, and post-translational modification.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

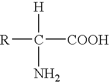

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, an “analog” of a chemical compound is a compound that, by way of example, resembles another in structure but is not necessarily an isomer (e.g., 5-fluorouracil is an analog of thymine).

The term “analyte”, as used herein, refers to any material or chemical substance subjected to analysis. In one aspect, the material is a peptide or mixture of peptides. In another aspect, the term refers to a mixture of biomolecules, including, but not limited to, lipids, carbohydrates, and nucleic acids such as DNA and RNA.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497). “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “basic” or “positively charged” amino acid as used herein, refers to amino acids in which the R groups have a net positive charge at pH 7.0, and include, but are not limited to, the standard amino acids lysine, arginine, and histidine.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biomolecule”, as used herein, refers broadly to, inter alia, a molecule produced or used by a living organism, or which is a substituent of a living organism. Biomolecules can be natural or synthetic. Biomolecules, include for example, but are not limited to, lipids, carbohydrates, proteins, peptides, and nucleic acids such as DNA and RNA.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a protein, polypeptide, an isolated nucleic acid, or other agent used in the method of the invention.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met, Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information NICBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “mass tag”, as used herein, means a chemical modification of a molecule, or more typically two such modifications of molecules such as peptides, that can be distinguished from another modification based on molecular mass, despite chemical identity.

The term “method of identifying peptides in a sample”, as used herein, refers to identifying small and large peptides, including proteins.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample.

As used herein, a “peptide” encompasses a sequence of 2 or more amino acid residues wherein the amino acids are naturally occurring or synthetic (non-naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl —C(O)NR— linkages (bonds) have been replaced by a non-peptidyl linkage such as a —CH₂-carbamate linkage

(—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

The term “peptide mass labeling”, as used herein, means the strategy of labeling peptides with two mass tag reagents that are chemically identical but differ by a distinguishing mass.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “polylinker” is a nucleic acid sequence that comprises a series of three or more different restriction endonuclease recognitions sequences closely spaced to one another (i.e. less than 10 nucleotides between each site).

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting group is such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A “recombinant polypeptide” is one which is produced upon expression of a recombinant polynucleotide.

A “sample,” as used herein, refers preferably to a biological sample from a subject, including, but not limited to, normal tissue samples, diseased tissue samples, biopsies, blood, saliva, feces, cerebrospinal fluid, semen, tears, and urine. A sample can also be any other source of material obtained from a subject which contains cells, tissues, or fluid of interest. A sample can also be obtained from cell or tissue culture. One of ordinary skill in the art will recognize that such a sample may comprise a complex mixture of peptides.

As used herein, the term “secondary antibody” refers to an antibody that binds to the constant region of another antibody (the primary antibody).

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds,” as used herein, is meant an antibody or compound which recognizes and binds a molecule of interest (e.g., an antibody directed against a polypeptide of the invention), but does not substantially recognize or bind other molecules in a sample.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured. Standard can also refer to a standard sample which is used for comparison to a test sample.

A “subject” of analysis, diagnosis, or treatment is an animal. Such animals include mammals.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C., with washing in 1× SSC, 0.1% SDS at 50° C.; preferably 7% SDS, 0.5 M NaPO₄, 1 mM EDTA at 50° C. with washing in 0.5× SSC, 0.1% SDS at 50° C.; and more preferably in 7% SDS, 0.5 M NaPO₄, 1 mM EDTA at 50° C. with washing in 0.1× SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

Methods useful for carrying out the present invention are described herein or are known in the art. Mason and Liebler described a quantitative analysis of modified proteins by LC-MSIMS of peptides labeled with phenylisocyanate (J Proteome Res, 2003. 2(3): p. 265-72). Mason did not describe complex peptide mixtures of ¹³C-phenylisocyanate. The reaction of PIC with amino terminal amines specifically has been known since the 60's. U.S. Pat. No. 6,908,740, entitled “Methods and apparatus for gel-free qualitative and quantitative proteome analysis, and uses therefore” [sic] describes (ordinary) PIC to neutralize charges on amines. They mention DS PIC in the context of mass tagging, but not ¹³C PIC or any strategies. They erroneously state that PIC reacts with all amines. U.S. Pat. No. 6,846,679 describes PIC as a capping compound that enables cleavage of capped amino acids. They mention the use of deuterated forms of capping compounds. U.S. Pat. No. 6,750,061 describes MALDI MS to analyze PIC-reacted peptides processed by Edman degradation for sequencing. Several other groups have characterized mass tagging reagents, most notably the ICAT and iTRAQ reagents sold through ABI (for example, see U.S. Pat. Nos. 6,982,414, 6,969,757, 6,963,807, 6,962,818, 6,944,549, 6,940,065, 6,931,325, 6,908,740, 6,900,430, and 6,872,574.

Useful methods include, for example, performing LC-MS/MS analyses, which can be performed on a ThermoFinnigan LCQ Deca ion trap MS instrument equipped with a ThermoFinnigan Surveyor HPLC pump and microelectrospray source and operated with ThermoFinnigan Xcalibur version 1.2 system control and data analysis software. Analysis of samples can be performed with an acetonitrile gradient and a Monitor C18 (Column Engineering) packed tip with 100 μm ID, 360 μm OD, and 5-15 μm tip opening. The flow from the HPLC pump can be split to achieve 500 nL to 1 μL flow rate from the packed tip. Two gradients can be used, “fast” and “normal”, depending on the complexity of the sample being analyzed.

As used herein, an “instructional material” includes a publication, a recording, a diagram, or any other medium of expression which can be used to communicate the usefulness of the invention in a kit. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

It will be appreciated, of course, that the peptides may incorporate amino acid residues which are modified without affecting activity or usefulness in the assay. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-ammo acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methyl amine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on then charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The invention is now described with reference to the following Examples and Embodiments. Without further description, it is believed that one of ordinary skill in the art can, using the preceding description and the following illustrative examples, make and utilize the present invention and practice the methods of the invention. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

### 13C (6) Phenyl Isocyanate, a Covalent Isotope Tag Specific for Peptide Amino Termini

Without wishing to be bound by any particular theory, it was hypothesized that a ¹³C labeled form of PIC might be useful for peptide mass labeling. A custom isotope synthesis lab (Isotech) was then contracted to prepare the desired ¹³C labeled PIC. The reagent was prepared and supplied in 99±% isotopic purity. The material was obtained in the pure state, and stored in anhydrous conditions at room temperature, or as a 100 mM solution in acetonitrile at −20°. Conventional ¹²C-PIC was obtained from Aldrich Chemicals.

Selective Amino-Terminal Labeling Conditions

The present experiments were designed to determine the specificity of PIC labeling of peptide amino termini, and sought to do so in complex peptide mixtures that have not before been analyzed using any isotopic form of PIC labeling. A sample of human cerebrospinal fluid (CSF) was digested with trypsin, then peptides were labeled with ¹²C-PIC, the labeled mixture was analyzed using LC/MS, then spectra were identified using SEQUEST. While labeling with PIC is robust, we have routinely labeled about 100 μg or less protein in 200 μl or 110 mM HEPES buffer pH 8, by adding either 2 or 4 μl of 0.1M PIC solution in acetonitrile. Care is used to reduce the presence of free amines in the reaction, and after 15 minutes the reaction is terminated by addition of 4 μl of 100 mM ammonium bicarbonate.

SEQUEST readily identified peptides from abundant proteins that were either unmodified or modified at amino termini or at lysine residues. Since roughly half or all tryptic peptides contain carboxy-terminal lysines, and some contain internal lysine residues resulting from incomplete tryptic cleavage, many peptides might potentially be labeled twice or more by PIC.

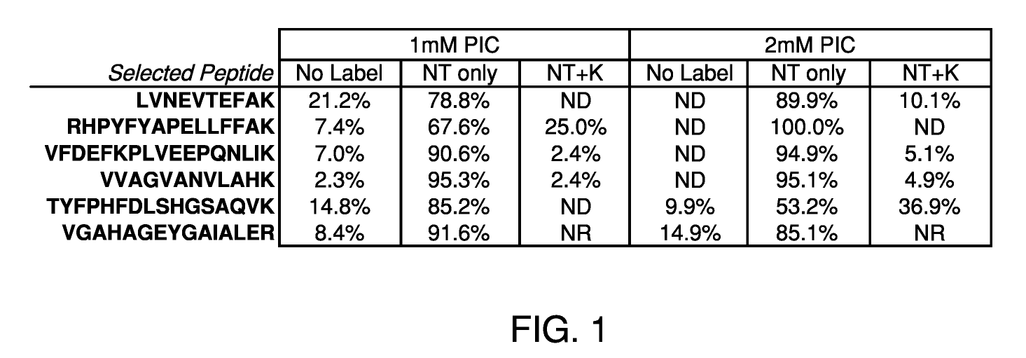

Spectra were selected that according to the SEQUEST analysis identified at high confidence several peptides that were modified by PIC at either the amino terminus or at internal lysine residues, as shown in FIG. 1. Quantification of these species based on MS peak height showed that the for the identified peptides showed about 90% modification on the amino terminus alone, 7% modification on both NT and lysine amines, and very low or undetectable modifications at the lysine residue alone or of the unmodified peptide, verifying the strong preference for predicted NT-labeling. In subsequent experiments we have been careful to use the same concentration and same PIC:peptide molar rations as in these pilot experiments, in brief this is incubation in amine-free pH 8.0 buffer containing 1 mM PIC and 1% acetonitrile for 15 minutes at room temperature, followed by quenching of reactive PIC with 10 mM ammonium bicarbonate.

Co-Elution of ¹³C-PIC and ¹²C-PIC Modified Peptides

Comparison of relative peptide abundance in LC/MS is highly dependent upon the lack of bias in the isolation of one labeled form versus another. Thus, co-elution of both peptides in chromatography is extremely important. To test whether differently PIC labeled peptides display identical chromatographic patterns, we again analyzed tryptic digests of human cerebrospinal fluid in a complex mixture. ¹²C-PIC has a monoisotopic molecular mass of 119.12, while ¹³C PIC has a mass 6 Da greater). Identical aliquots of trypsin-digested CSF proteins were labeled with ¹³C-PIC and ¹²C-PIC, to compare the behavior of a wide range of peptides labeled with these two tags.

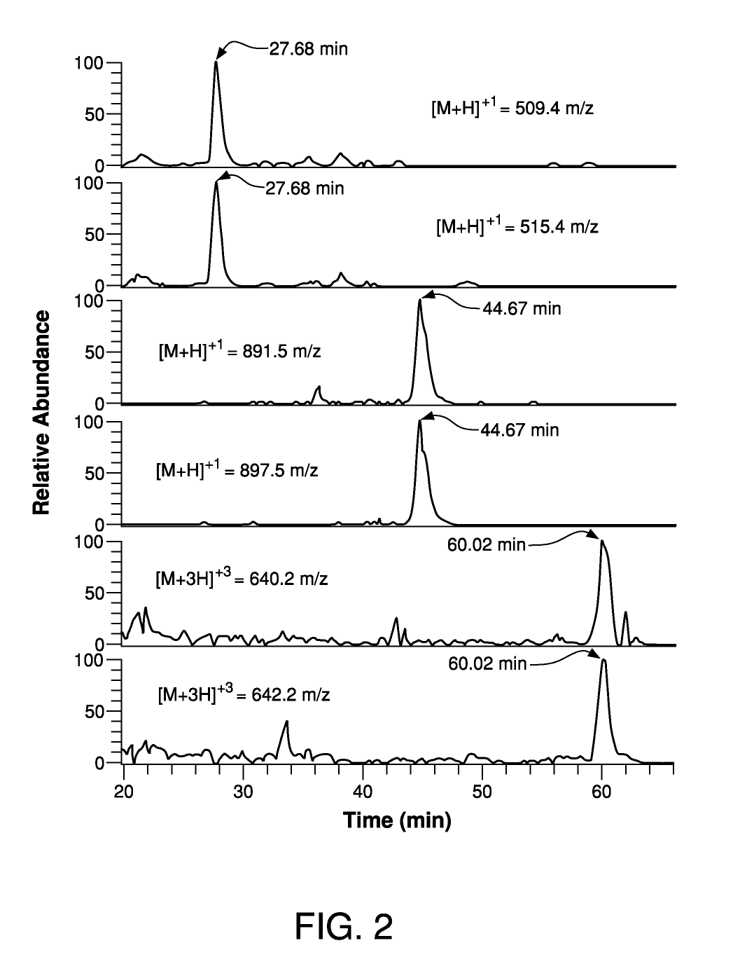

Examination of individual LC/MS spectra identified many examples of peptides labeled with either PIC-L or PIC-H, that were evident as pairs of ions separated by exactly 6 m/z, and will be discussed below. Comparison of the ion chromatograms of all of these peptide pairs showed exact coelution from the reversed phase nanochromatography column, to a precision of elution peaks identical within 0.01 minute. Several of these ion chromatograms are shown in FIG. 2, which are typical of the chromatographic behavior of all peptide pairs we have examined.

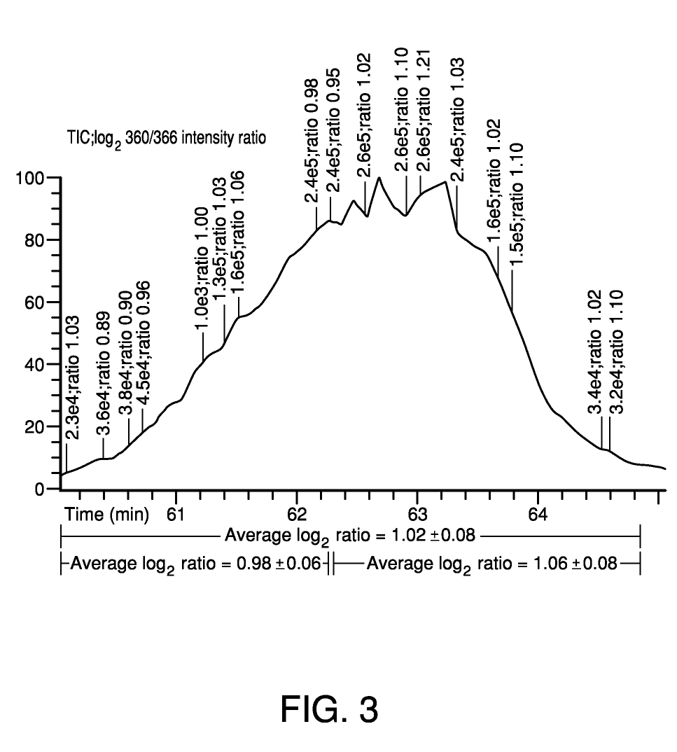

Because co-elution might be subtly different between two mass-tagged pairs, quantification experiments are at risk of overestimating the abundance of one tagged species at the beginning of an elution profile (if that tagged protein elutes slightly before the other) while underestimating the abundance near the end of the elution profile. Sampling algorithms for LC/MS instruments typically attempt to sample abundant ions infrequently, in order to sample the maximal number of less abundant ions. It was found, however, that a very abundant ion pair of 360/366 m/z was sampled 17 times during our run, beginning when the elution peak was just beginning. The C¹² to C¹³ ratio was compared, theoretically 1.0, at each of the times at which this pair was identified (see FIG. 3). Over a 4.5 minute period, in the middle of the elution gradient, and across an intensity range from 2.4×10⁴ to 2.6×10⁵, it was found that the log₂ concentration ratios very nearly equaled the theoretical 1.0, being an average of 1.02±0.08.

Strategies for Quantitative Analysis of Isotope-Tagged Peptide Pairs.

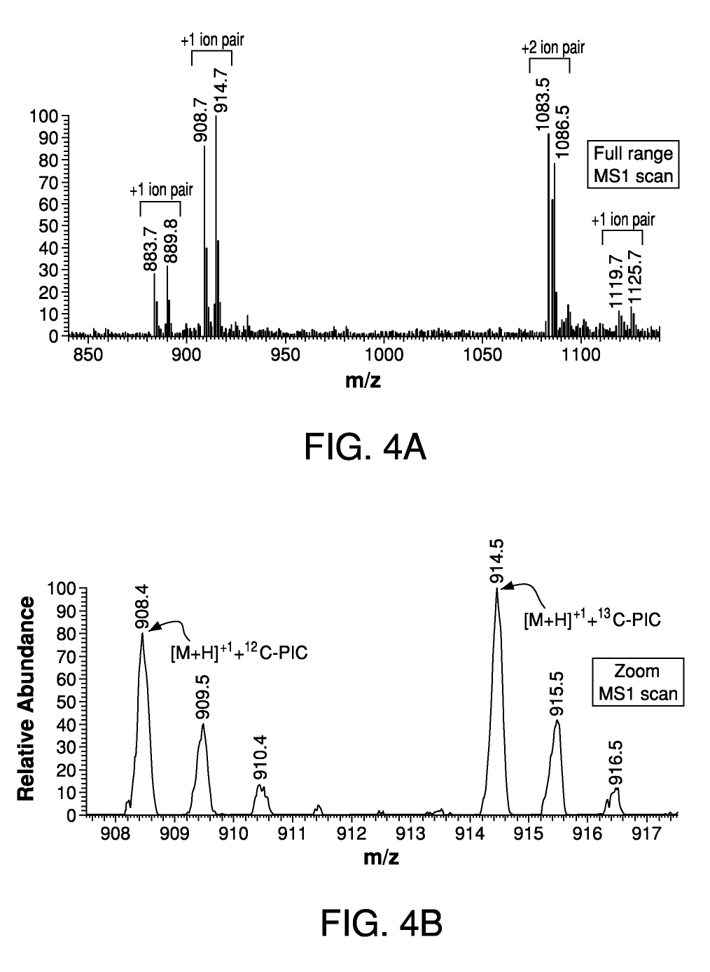

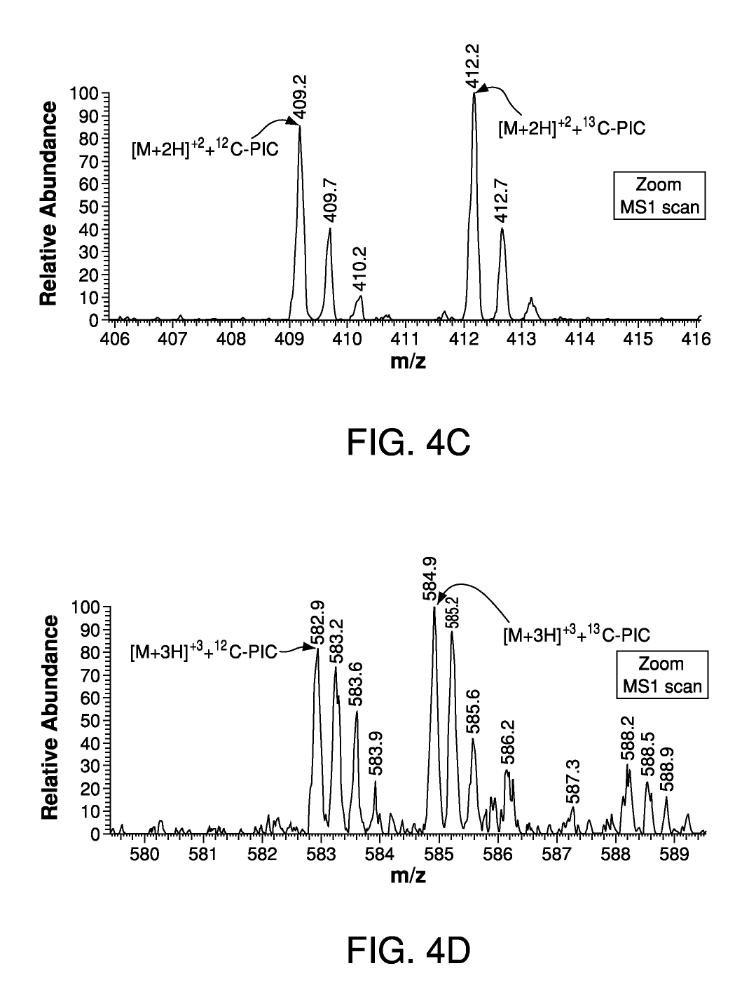

Strategies for quantification of isotope-tagged peptide pairs remain unoptimized. At its simplest, mass tagging with PIC results in pairs of otherwise identical masses that differ by a mass of six Da supplied by the ¹³C atoms in the ¹³C PIC. Ions with a charge of +1 should differ by precisely 6 m/z while ions of +2 and +3 charge will differ by 3 and 2 m/z units respectively. Examples of each of these ion pairs are shown in FIG. 4.

An optimized data collection protocol was devised during LC/MS/MS, where an initial MS1 spectrum is acquired by the instrument over a wide mass window, and the machine is programmed to select high intensity ions for subsequent analysis. Similar to other MS routines, the instrument is programmed to record the mass of recently sequenced ions in order to reduce the repetitive acquisition of previously identified ions. In the modification disclosed herein, to optimize precise measurements of both mass and intensity, the instrument was programmed to acquire first the wide range MS1, then to collect paired sets of spectra-first a narrow range (15 m/z) “zoom” MS1 scan of the selected ion, and then an MS2 scan of the same target ion. Between 6 and 30 spectra are selected for analysis before a new wide scale MS1 scan is taken. In this way, a complex mixture analysis might result in acquisition of 1500 wide scale MS1 scans, 15,000 zoom MS1 scans, and 15,000 MS2 scans. In practice, information from the zoom and MS2 scans are analyzed together, with precise mass and peak quantification derived from the zoom scan and neutral loss and fragmentation derived from the MS2 scans.

The use of zoom MS1 scan to quantify ion peaks has significant advantages as shown in FIG. 4, over the use of wide range MS1 scans. Significantly, an ion that is segregated into two adjacent electronic bins might have an apparent intensity almost 50% reduced from the theoretical intensity if the ion was captured exclusively into one bin. The zoom MS1 scan, by providing many more bins surrounding the target ion range, ensures that peaks of similar shape are accurately quantified relative to each other on the basis of peak height determination (FIG. 4, Panels B-D).

Manual and Automated Analysis of PIC Labeled Spectra.

Presented below are several approaches through which spectra can be identified and quantified by analysis of PIC-labeled spectra.

An Improved Data Acquisition Strategy for Quantification of MS1 Peak Heights

The significance of using zoom scan determinations as opposed to wide range determinations was validated by examination of 42 ion pairs derived from an identical complex protein sample divided in two and then labeled separately with ¹³C or ¹²C PIC, then mixed. Forty-two peptides that were identified by SEQUEST database searching were quantified by wide range MS1 scans or by peak height determination from zoom scans (Table 1). While the average ratio of ¹³C or ¹²C was about equal to the theoretical ratio of 1:1, the ratios derived from the wide range scan had a standard deviation of almost 1, while those derived from zoom scan determinations had a standard deviation of only 0.14. Thus, the precision of zoom scan analysis is far greater than that of wide range MS1 quantification. While these quantifications were performed manually and individually using data from single scans, this process was automated using the PICquant software as described below.

An Improved Strategy for Identifying Quantitative Alterations in Complex Peptide Mixtures: Quantify-then-Identify

Overall, the PIC label strategy and the ability to quantity peptide ions rapidly has enabled the pursuit of a new approach to comparative quantification of complex peptide mixtures. Current automated strategies for complex mixture analysis first identified peptide sequences corresponding to peptide spectra through database searching, and then attempt to quantify the identified spectra. This approach has two drawbacks, one is that database identifications are frequently inaccurate, necessitating manual corroboration which is impractical for thousands of spectra. Secondly, database search algorithms identify spectra with high-confidence for only a small minority (about 1%) of all spectra. This means that the vast majority of spectra that do not receive identification are not tested for quantitative differences.

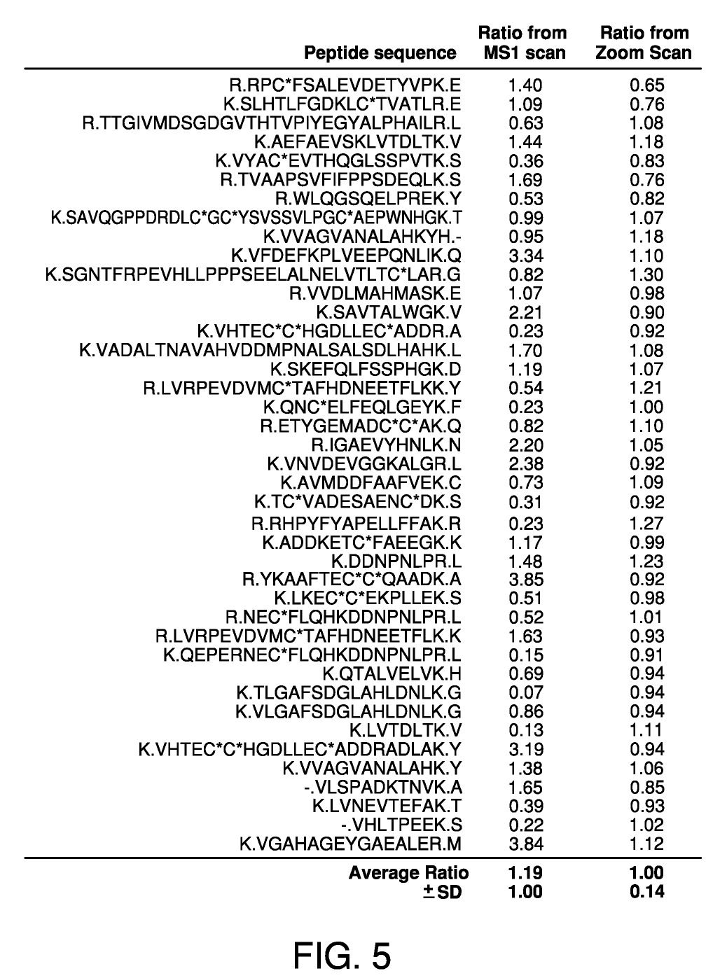

The revised strategy, as diagrammed in FIG. 5, is to first quantify all available spectra, identifying those that differ between ¹³C or ¹²C labeled spectra quantitatively by more than a specified ratio, i.e., 2 fold or 5 fold. Subsequently, only these few dozen or hundred spectra are used to search the database, and identities are confirmed by manual analysis. While the concept of “Quantify then Identify” might prove useful in several labeling strategies, the use of PIC labeling significantly enables quantification because it clearly identifies which spectra demonstrate quantitative differences.

Use of PIC Label to Identify Charge and Label Status of Raw Spectra

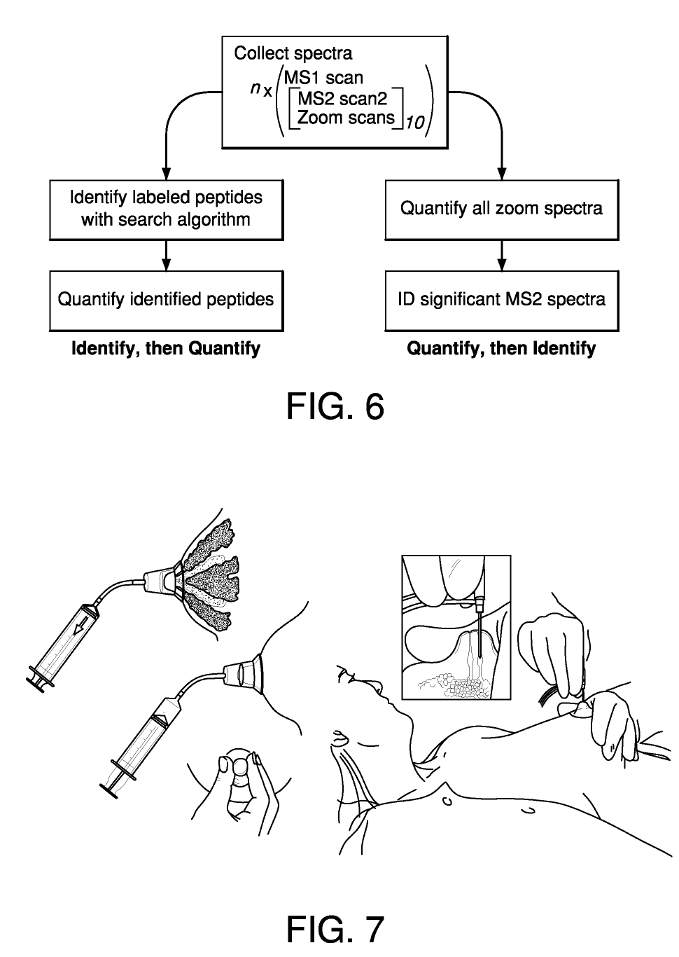

To test the use of PIC in a real world situation, the technique was applied to clinical specimens derived from bilateral breast ductal lavage of women with unilateral breast carcinoma. Thus, one specimen in concept might contain cancer-derived proteins that are absent from the alternate specimen, while other ductal epithelium-derived proteins might be common to both specimens (see FIG. 6). We digested aliquots of protein from both specimens and labeled them with ¹³C or ¹²C PIC, then combined them for analysis on a single LC/MS run using the protocol described above.

It was expected that pairs of PIC-labeled peptide-derived ions would be observed and could be quantified using the strategies described above. It was predicted that peptides derived form proteins present in one specimen and not in the other would be evident as singlet peaks that did not have a corresponding partner peak. While examining spectra from this experiment it became clear that we could not directly distinguish differently-expressed peptides represented by singlet peaks from singlet peaks resulting from peptides that escaped PIC labeling. Because it was expected that unlabeled peptides might represent 5-10% of all peptides in the mixture, these decoy singlets would likely obscure the true differentially-unique peptides. From this, it became clear that a method was needed to distinguish unlabeled peptides from PIC labeled peptides using information from MS2 spectra.

A Neutral Loss Signature Characteristic of PIC Labeling

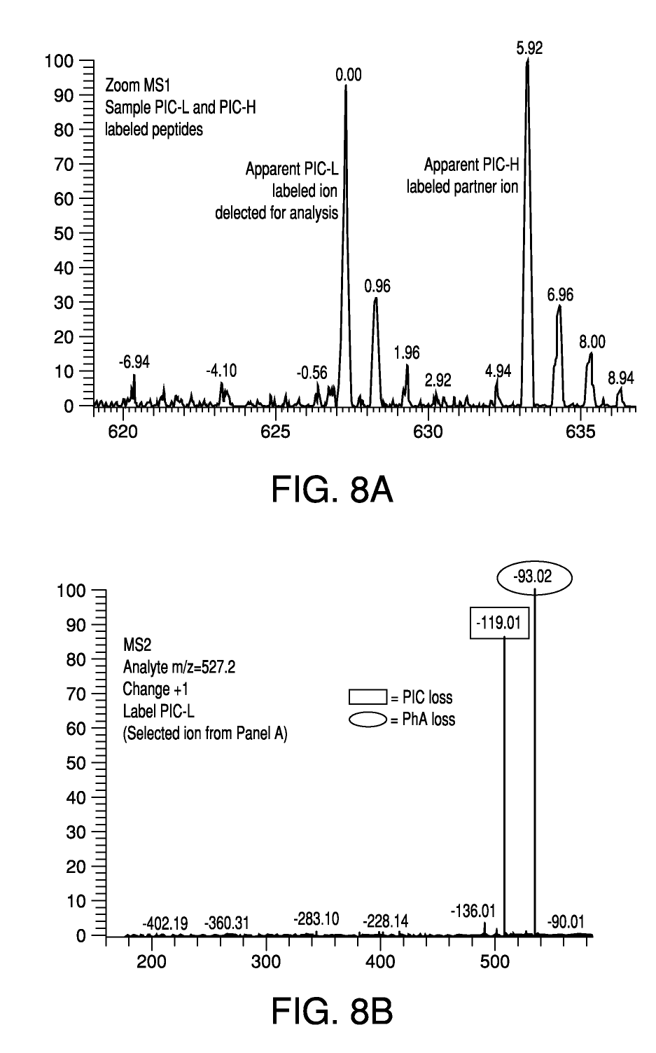

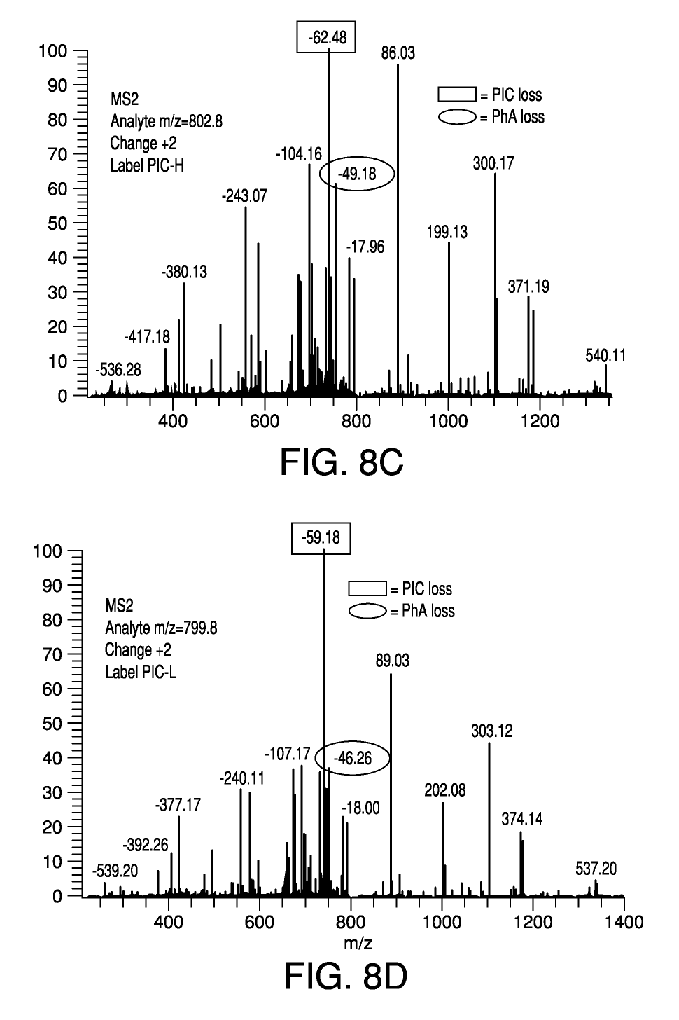

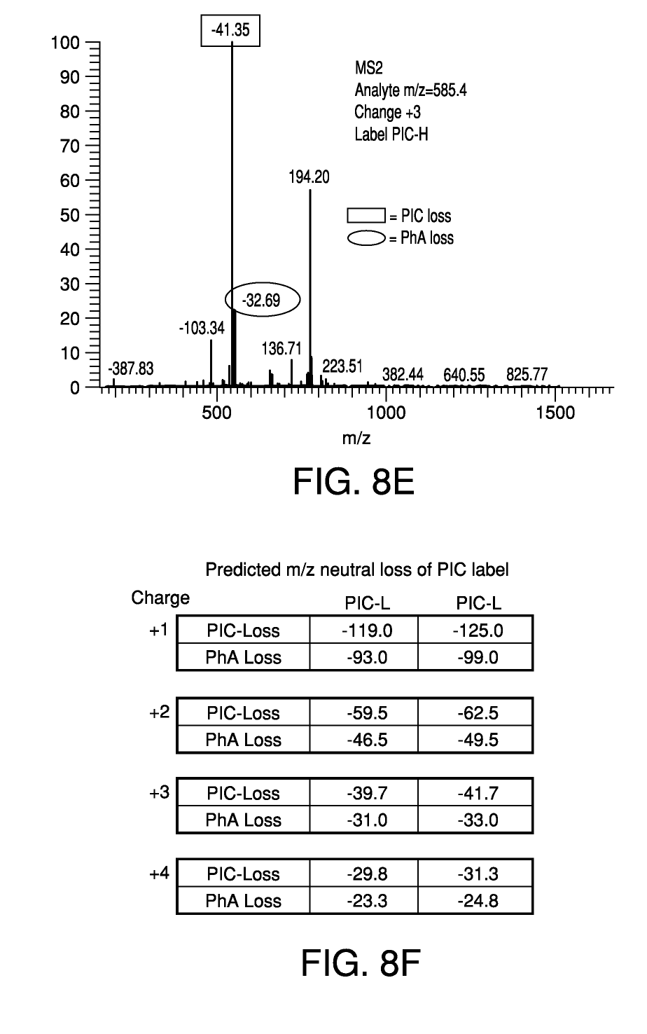

This problem above was resolved by analysis of spectra from ion pairs in this experiment, as shown in FIG. 8. Panel A shows a well-resolved ion pair separated by 6 m/z units, resolved on MS1 ‘zoom’ scan, and thus apparently of +1 charge. In analysis of the corresponding MS2 spectrum derived from the ¹²C PIC labeled 627.25 m/z precursor peptide, the mass of the MS2 ions was examined relative to the mass of the precursor ion (Panel B), displaying of the MS2 fragment peptide ions by masses relative to the mass of the precursor ion.

PIC-Labeled Peptides Displayed Two Characteristic Neutral Loss Ions that Distinguish PIC Labeled Peptides from Unlabeled Peptides.

Specifically, MS2 ions were observed with neutral loss of −119 m/z (compatible with the loss of ¹²C-PIC label itself, and labeled in FIG. 8 “PIC loss”) were seen in the MS2 spectra. A second pattern of neutral loss was also seen that had a neutral loss of −93 m/z, which is compatible with an ion that loses the phenyl group and oxygen of ¹²C-PIC label, but retention of the isocyanate moiety with the peptide (termed “PhA loss”). These signature neutral loss ions differ for peptides labeled with ¹³C or ¹²C PIC. For example, for a +1 charge peptide, ¹³C PIC label results in ions with relative loss of −125 and −99 m/z, while ¹²C PIC labeled peptides yield ions with neutral loss of −119 and −93 m/z. Of note, these neutral loss masses are distinct from any natural amino acids could otherwise result in similar product ions.

It was recognized that peptide ions of different charges would give rise to neutral loss products of a different m/z, which were observed in virtually all MS2 spectra of PIC-labeled peptides, for which examples are shown in FIG. 8 panels C, D, and E. To simplify identification of these distinctive neutral loss fragments a diagnostic table (FIG. 8, Panel F) was created that shows the predicted m/z loss for peptides labeled with ¹³C PIC or ¹²C-PIC, and bearing charges between +1 and +4. This table (FIG. 8, Panel F) has proven to be extremely informative because, when used to analyze MS2 fragmentation spectra, MS2 ions matching these predicted loss ions uniquely identify both the charge state of the ion and the label (¹³C or ¹²C) of the PIC. Additionally, peptides lacking these characteristic neutral loss fragments are inferred to be unlabeled, and thus ignored in the analysis of peptide pairs.

PIC-Assisted Interpretation of Spectra Identifying Quantitative Differences in Complex Peptide Mixtures.

In practice, using the table in FIG. 8 manual analysis of PIC-labeled spectra follows an optimized pattern: MS2 spectra are first examined for these characteristic neutral loss ions, and the charge and label type noted. Then, examination of the MS1 spectrum is simplified, since the charge and label type enables prediction of the m/z of the predicted partner peptide for analysis. Finally, relative expression of the ¹²C- and ¹³C-PIC labeled peptides is determined by peak height.

FIG. 8 presents several examples of spectrum sets that identify quantitative differences between ¹²C and ¹³C labeled peptides from the ductal lavage experiments. In these ions, MS2 spectra revealed PIC and PhA neutral loss ions that enabled charge and label type identification, then singlet ions observed in ‘zoom’ MS1 scans were annotated (arrows) with the predicted location of the partner ion. In these four examples, abundance of partner ions varied by 4-fold or more, indicating a greater abundance of peptide in one of the original samples than in the other.

The examples displayed in FIG. 8 were selected from several dozen because these spectra were all were identified using the SEQUEST algorithm as deriving from the same original protein of approximately 70 kDa. The identity of this protein is not relevant to the current application, but the presence of multiple peptides provides increased certainty that a genuine abundance difference in the original clinical samples underlies the difference observed in the spectra.

Automated Analysis of Peptide Abundance Using PICquant

A computer based algorithm was written that scrutinizes MS2 fragmentation in a manner that emulates the manual quantitative analysis presented above. Specifically, MS2 spectra are examined for the presence of ions with distinctive neutral losses predicted from this in FIG. 8. The type of neutral loss ion identified determines the charge state of each selected ion, and also predicts whether it is labeled with ¹³C or ¹²C PIC. Typically, PICquant identifies somewhat more than half of all MS2 spectra as being PIC labeled; others spectra derive from unlabeled peptides or have more than one characteristic neutral loss peak, likely signifying a mixed peptide.

Secondly, PICquant predicts the m/z of the partner ion, and then quantifies the expression of both ¹²C and ¹³C ion pairs automatically, and returns a table of this information, which is usually sorted to highlight spectra that show varying ¹²C/¹³C PIC labeled peptide ion abundances.

PIC Labeled Peptides have Improved Information for Sequence Identification

In addition, PICquant determines additional information from the spectra. First, the mass of the analyzed (target) ion determined with greater accuracy, either from the high-resolution zoom scan or from the mass of the “-PhNCO loss” ion. Both zoom MS1 and MS2 spectra display far greater precision than wide range MS1 spectra. Improved mass resolution and definite ion charge state enable more precise analysis of peptide identifications using SEQUEST and other search algorithms. Thus, the PIC labeling strategy will improve the likelihood that spectra receive accurate sequence identification.

PIC Labeled Peptide Spectra Enable Signatures of Peptides in their Unlabeled State, for Proteomics Meta-Analysis

Importantly, the mass of the target peptide before PIC labeling can be calculated either manually or using PICquant. This is done by subtracting the mass of ¹²C PIC or ¹³C PIC as indicated from the mass of the peptide determined by zoom scan, or alternatively by measuring the absolute mass of the signature “-PhNCO” peptide described above. This calculation results in what we term the “root mass” of the peptide. Importantly, the root mass of a ¹³C PIC-labeled peptide is the same as the root mass of a ¹²C PIC labeled peptide (because the peptides were the same before labeling). This root mass is important because it is an identifying feature of a peptide that not only might show quantitative features in the original experiment, but that can also be used to identify peptides in unrelated experiments so that expression profiles between experiments might be compared.

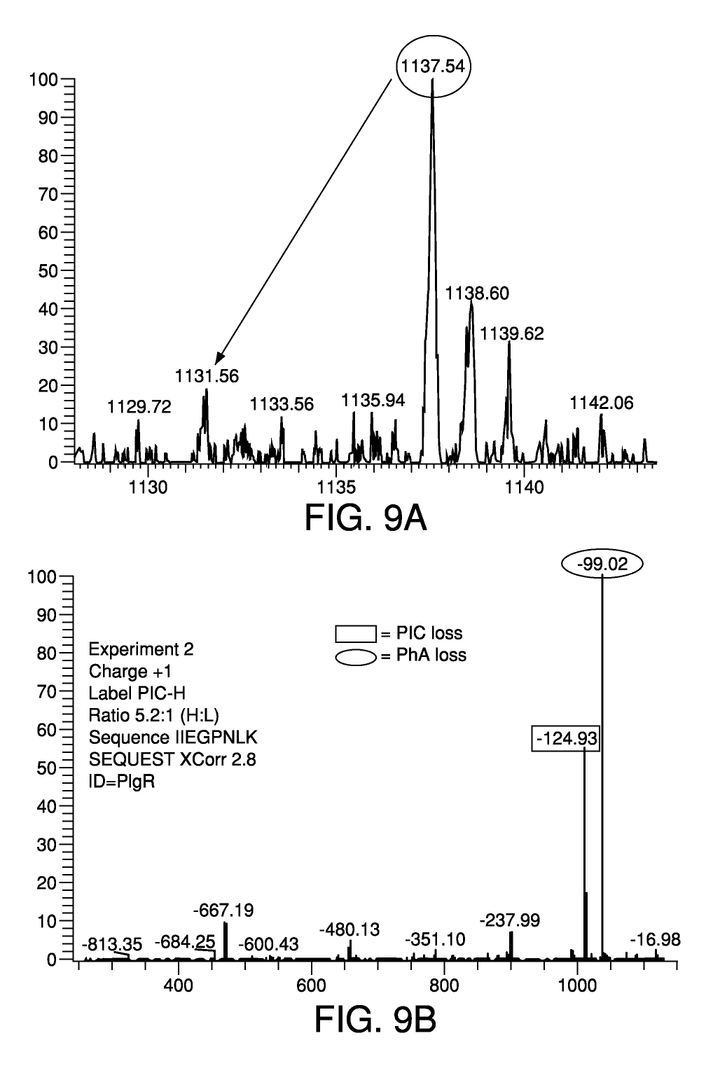

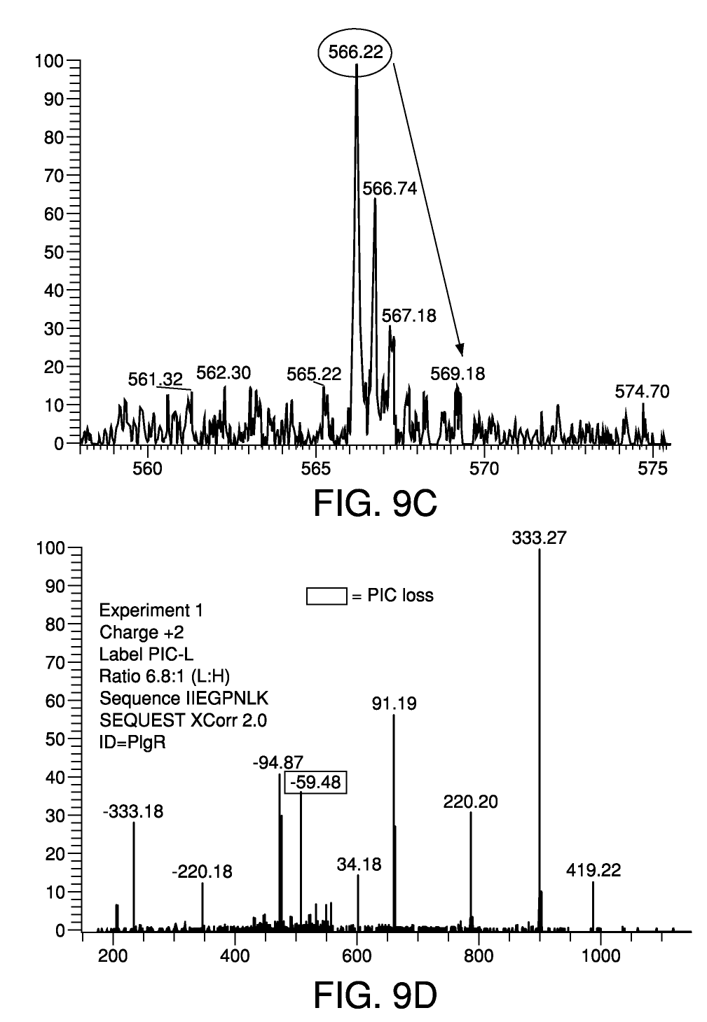

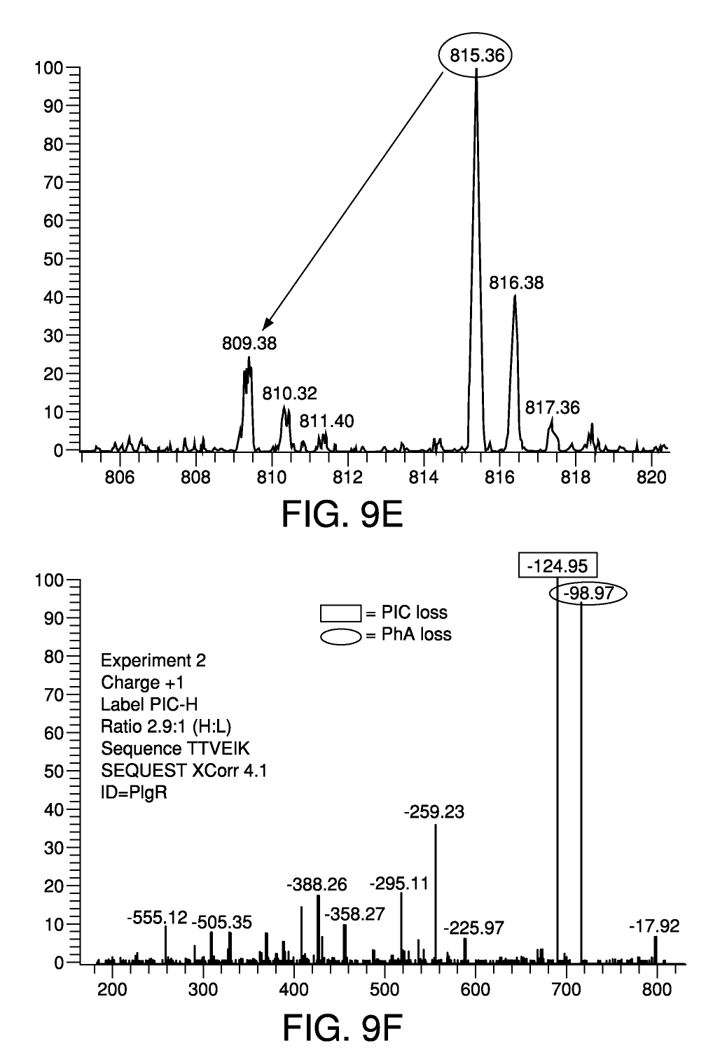

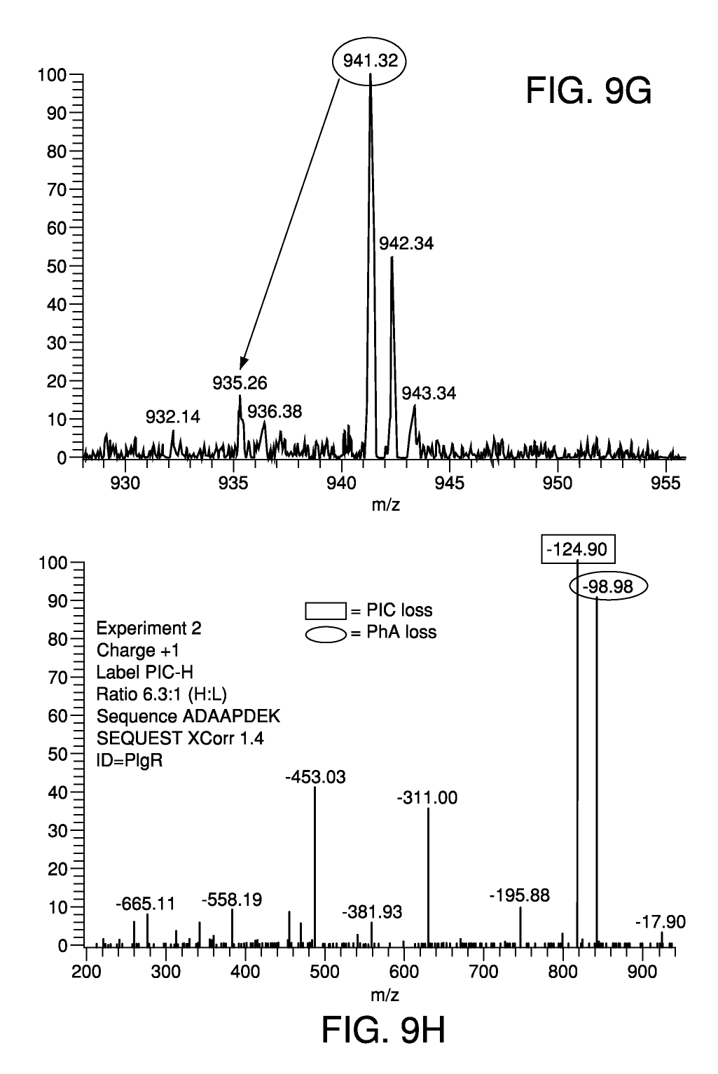

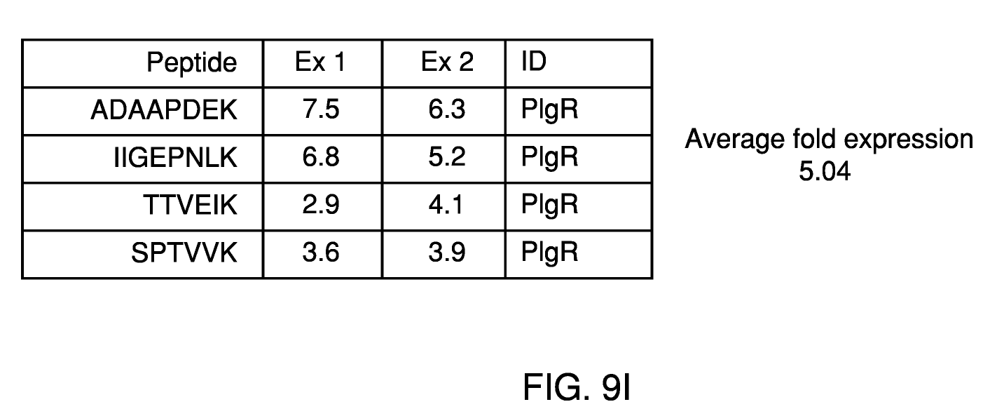

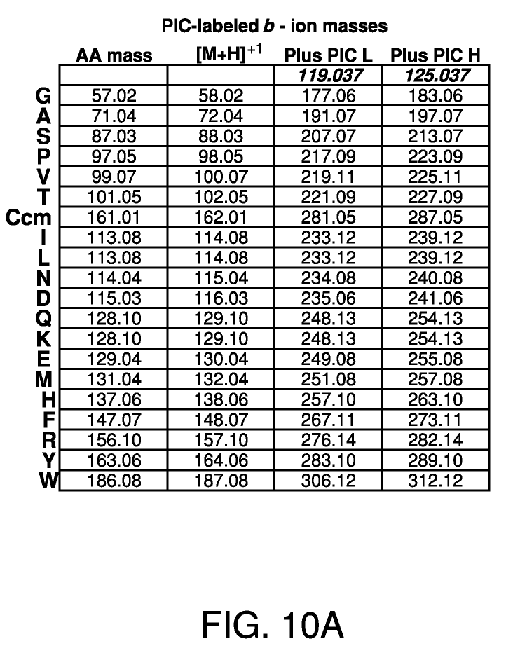

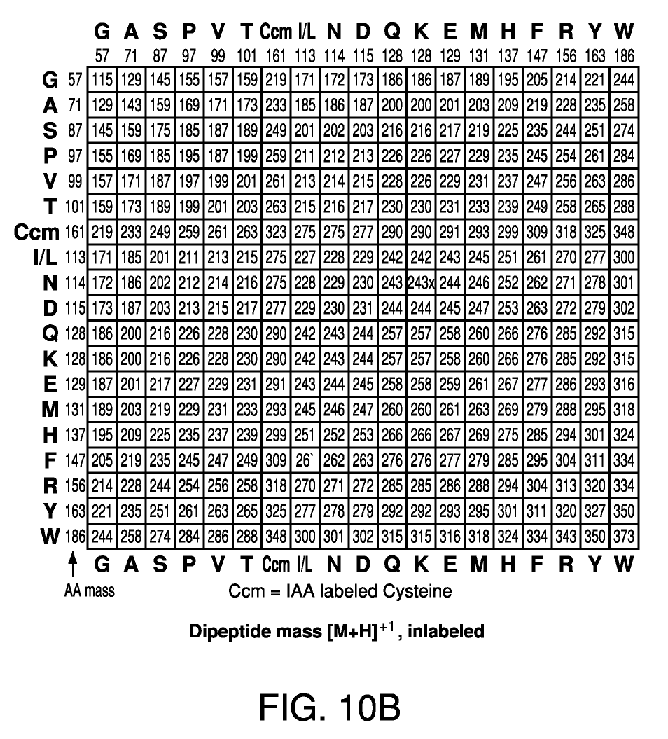

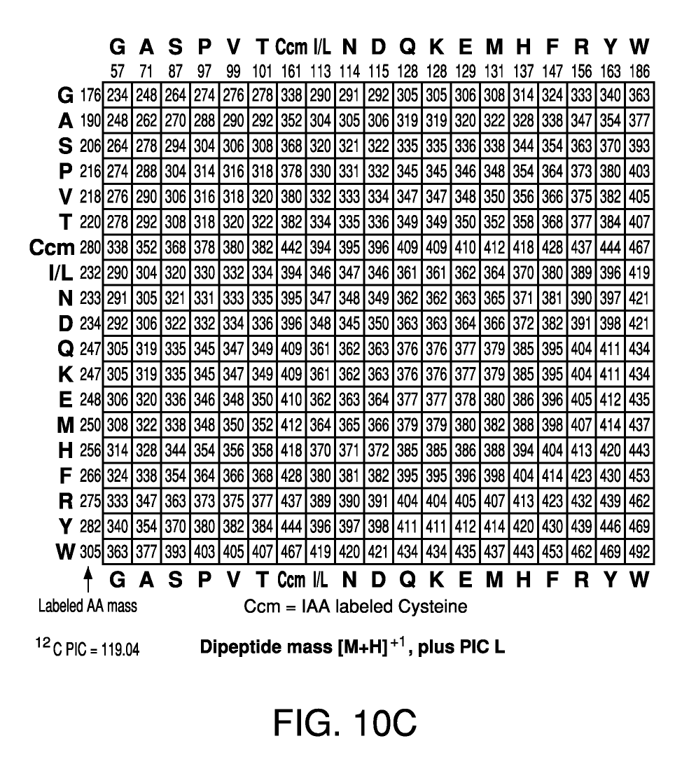

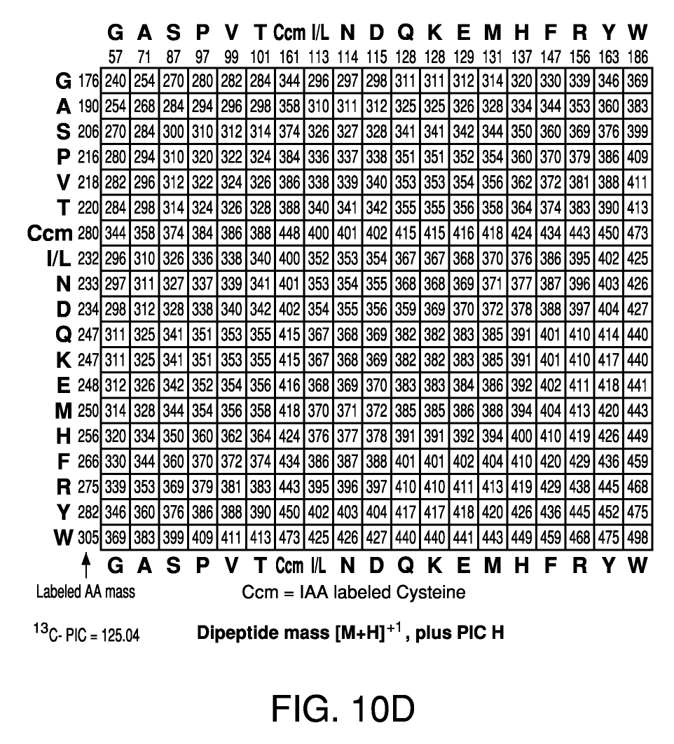

For example, if an ion of 1137.54 (FIG. 9A) is observed that displays quantitative differences among peptides in an initial experiment, the root mass of this +1 charge, ¹³C labeled peptide can be calculated as (1137.5-125.04) or 1012.50 Da. Other spectra can be identified with a root mass of 1012.5, that are likely to be the identical peptide. These peptides can then be quantified by examination of their zoom spectra. Importantly, experiments performed on different days or in different labs can also be examined using spectra that indicate a peptide with the same root mass, and these spectra can be quantified. In this way, an informative meta-analysis can be performed comparing dozens to thousands of independent experiments in which quantitative analysis of PIC-labeled peptides can be compared.

The value of the data thus derived and compared expands dramatically with the number of samples analyzed. The PICquant program stores data and calculated results in a SQL database, thus easily enabling direct comparison of result from several parallel experiments. Given an average of 70 MB of data storage requirements, data from over 4000 individual experiments can be stored on a single 300 GB hard drive.

PIC Labeling Enhances Accuracy of Database Search Algorithms

Sequence identification using database search algorithms is limited in part because of algorithm-generated errors in the assignment of charge and mass determinations to individual spectra. The PIC labeling strategy greatly improves the assignment of both mass and charge of the peptide underlying the spectrum under examination, and thus enhances the speed and accuracy of these algorithms.

Improved Mass and Charge Determinations

As mentioned above, accurate peptide masses were derived using our data acquisition algorithm in two ways. First, zoom scan spectra provide accuracy within 0.05 m/z units. Secondly, this mass determination was confirmed using the MS2 spectra, specifically as the mass of the “-PhNCO” peak shown in FIGS. 8 and 9. The accuracy of these mass determinations was also in the range of 0.05 m/z units. The degree to which this improved mass determination improves sequence identifications remains to be calculated, but at least some improvement is certain. Secondly, the data acquisition and analysis algorithms provide accurate charge determinations, using the data in the table in FIG. 8F. Because this approach requires accurate (less than 0.1 m/z unit) agreement with two different neutral loss fragments, the accuracy is expected to be very high. Precise accuracy has not yet been calculated, but to date, not a single spectrum has been misidentified, when comparing manual to automated charge analysis. Conventional charge identification using current database search algorithms quite inaccurate. As an example, the SEQUEST algorithm often returns identified protein candidates with similar confidence, even though the identification is made based on two different charge states!

To take advantage of these improved data features disclosed herein, input data files are created that contain the improved data. For example, in the Finnegan data platform, the “.dta” (data) files contain spectral information, coupled with empty or approximated fields for m/z and charge, which are subsequently filled using the SEQUEST algorithm. With the platform of the invention, a script called “DTAout” constructs synthetic .dta files that contain highly accurate m/z and charge state determinations. Collections of these files are then used to search peptide databases using SEQUEST.

Improved Fragment Ion Identification

Database search algorithms operate by matching or correlating actual MS2 fragmentation spectra to theoretical spectral ions that are predicted by the databases. In fragmentation sequencing, peptide fragments are observed that come from both the amino terminus (termed b-ions) and from the carboxy terminus (termed y-ions). Importantly, it is very difficult to determine a priori which observed ions in an MS2 spectrum are b-ions and which are y-ions. This uncertainty greatly increases the difficulty of obtaining an accurate identification.

The PIC labeling strategy presents a unique opportunity to assist identification because it enables discrimination of b-ions and y-ions. This is enabled through two means, largely because of the specificity of the PIC mass tag to covalently modify proteins, mainly on the amino terminus.

Identifying Amino-Terminal Peptides by Calculated Peptide Tables

Because the mass of both ¹²C and ¹³C labeled PIC is unlike any natural amino acid, an N-terminal amino acid labeled with PIC has a mass unlike any other dipeptide combination that might be identified in a MS2 fragmentation spectrum. The tables shown in FIG. 9 demonstrate these points. In FIG. 10A, a table displays the masses of simple amino acids (column 1) and the same amino acids bearing a proton (i.e. charge +1, column 2). In a conventional MS2 spectrum, identifying this mass would indicate this amino acid as an amino-terminal, or possibly carboxy terminal, amino acid residue. In columns 3 and 4, the calculated masses of these same amino acids labeled with ¹²C or ¹³C (“PICL or PICH”) respectively. MS2 fragmentation spectra containing these ions are diagnostic of a peptide containing the indicated amino acid modified with PIC. In conventional analysis, masses in this range might indicate dipeptides from the amino or carboxy terminus. However, no natural dipeptides match the mass of a PIC labeled N-terminal amino acid. The calculated (+1) dipeptide masses is shown in the table in FIG. 10B for comparison.

A PIC-labeled amino acid is the smallest of possible amino-terminal B-ions. In a similar fashion, PIC-labeled dipeptides have (in most cases) unique masses that differ from other di- and tri peptides. These calculated PIC-labeled dipeptides are shown in FIG. 10C (for ¹²C-PIC-labeled peptides) and FIG. 10D (for ¹³C-PIC labeled peptides). Identification of ions in MS2 fragmentation spectra matching one of these predicted masses is strong evidence for the amino-terminal sequences of the parent peptide. Other ions that do not match these calculated masses are most likely

Identifying b-Ions and v-Ions Through Ms3 Fragmentation Analysis

As stated above, MS2 fragmentation spectra of PIC-labeled peptides such as those shown in FIGS. 8 and 9 display distinctive neutral loss peaks termed “-PhA” and “-PhAmine” herein. Complete loss of the PIC label is termed “PIC loss”. Importantly the “-PhNCO” ion is derived from a peptide identical to the natural peptide before PIC labeling. We have programmed our spectrophotometer to capture and fragment ions that match the “-PhNCO” ion, and obtain an “MS3” fragmentation spectrum from these selected ions. In this way, comparison of PIC-labeled peptide MS2 spectrum to the MS3 spectrum derived from the “-PhNCO” ion will show identical ions derived from carboxy-terminal peptides, and distinct peptides derived from amino-terminal peptides. This is because the two parent peptides differ only by the amino-terminal PIC label.

A computer script entitled “IonSort” compares spectra from selected sets of MS2 and Ms3 spectra, and defines likely b-ions and y-ions, and segregates these two ion types into separate “.dta” files containing only one type or the other, which can then be searched using at least some search algorithms (for example OMSSA). While the amount of data in each of these file types is reduced by about half from the original MS2 spectrum, there are fewer irrelevant ions that can serve as decoys to the peptide identification algorithm. Equally importantly, the b- and y-ion spectra are much more easily interpreted by manual analysis, in many cases enabling direct peptide sequence determination.

PIC Labeling for Assistance in Identification of Post-Translational Modifications

Posttranslational modifications of proteins are common results in alterations in cells and organisms under disease conditions including cancer. Typical modifications include phosphorylation; acetylation, methylation, proteolysis, and other physical changes of one or more amino acids and thereby change the function of the protein. Identification of protein modifications by mass spectroscopy usually involves identifying tryptic peptides that display mass modifications typical of the amino acid modification. However, the large number of possible peptides, the presence of contaminating proteins, and the possible low stoichiometry of modification makes identification of specific modifications difficult at times.

In principle, identification of specific modifications in peptides is a problem similar to identification of quantitative differences of peptides in complex mixtures. To address this problem, it is helpful to be able to isolate or partially purify the protein in question under two conditions, one in which the protein modification is maximally present, and another in which the modification is absent. Examples of such conditions include: a phosphoprotein treated or not with phosphatase in vitro; a methylated histone isolated from growing or quiescent cells; a full length protein compared to a proteolyzed protein; a protein analyzed on an SDS-PAGE gel that displays a mobility shift resulting from protein acetylation, and others.

In PIC-enabled identification of protein modifications, these protein pairs are isolated under the two conditions, for example by excising two protein gel bands on SDS-PAGE, one from each growth condition. Complete purification is not required in this situation, but it is helpful if proteins that might contaminate the proteins are similar in both preparations. When using conventional approaches, proteins within the gel band are digested to extract tryptic peptides. For PIC-assisted analysis, digested peptides are reacted separately with ¹³C- or ¹²-C PIC, and then mixed together for LC/MS analysis.

In a conventional analysis, these preparations would be analyzed separately using MS approaches, where spectra are searched against protein databases, or more specifically against a dataset including only the protein in question. Similar to biomarker discovery experiments this step is problematic. Overall, it is to be expected that 90% or more of spectra will not match the target protein with high reliability. The conventional approach thus may fail to identify a modification even if spectra are obtained from the modified peptide. The problem is of course amplified if the nature of the modification is not know, so that search algorithms cannot be directed to allow for a specific modification.

A different “quantify then identify” approach, or rather in this case “identify the spectrum, then identify the modification” approach is enabled using the PIC-labeled peptide mixture. Using the strategies described above, differences between the ¹³C- and ¹²C-PIC labeled peptides can be identified. In theory, spectra from the involved peptide should be found that is modified with only one of the PIC labels while all other peptides, including both contaminating proteins and unaffected parts of the target protein, should be equally labeled by both ¹³C and ¹²C PIC. After identification, the involved peptide can be identified with a combination of manual and computer-assisted approaches.

Significantly, knowing the nature of the modification is not necessary using this approach. Instead, examination of PIC-labeled spectra (as described above) can reveal b- and y-ions that reveal both N- and C-terminal sequences. This will in most cases result in precise identification of the peptide in question, if the complete amino acid sequence of the protein is known. From this, the theoretical mass of the unmodified peptide can be calculated, and compared with the root mass of the PIC-labeled peptide, determined as described above. This comparison will produce the mass of the modification, for example 80 Da for phosphorylation.

In most cases, particularly with short peptides, the amino acid sequence of the natural peptide can be read easily from the spectra until a gap is detected with a residue of a larger than anticipated mass is detected. While even PIC-guided spectrum interpretation requires a logical analysis, it is highly simplified by the reliable identification of an affected ion spectrum that does not require sequence identification of the modified peptide.

### SUMMARY

The isotopes-tagged PIC labeling reagent and subsequent analysis routines represent an approach that offers significant improvements in quantification of complex mixtures for identification of potential biomarkers. ¹³C-PIC labeling offers the following advantages: 1) it labels nearly every peptide in the mixture; 2) it presents a simple modification pattern, with in most cases single modification events; 3) it is indiscriminate in that it labels almost all peptides; 4) it results in distinctive chemical properties to labeled peptides that enable automated characterization of raw, unidentified spectra; and 5) it enables identification of labeled b-ions to enhances spectra interpretation and identification.

The present invention, consisting of the use of this novel and unique mass tag label, as well as schema and algorithms to interpret labeled peptides and proteins, significantly enhances the current state of the art of protein analysis and quantification by mass analysis methods. Application of the methods of the invention will enable discovery of protein modification and expression differences, which will in turn enable discovery and disease diagnostics.

Other methods which were used but not described herein are well known and within the competence of one of ordinary skill in the art of cell biology, molecular biology, and medicine.

The invention should not be construed to be limited solely to the assays and methods described herein, but should be construed to include other methods and assays as well. One of skill in the art will know that other assays and methods are available to perform the procedures described herein.

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety. While this invention has been disclosed with reference to specific embodiments, it is apparent that other embodiments and variations of this invention may be devised by the previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

1-9. (canceled)

10. A method for identifying at least one peptide in a sample, said method comprising;
obtaining a first sample;
contacting an aliquot of said first sample with ¹³C-PIC, thereby labeling at least one peptide with ¹³C-PIC in said aliquot;
analyzing said aliquot to validate ¹³C-PIC-labeling of at least one peptide;
obtaining a second sample, wherein said second sample comprises an otherwise identical sample to said first sample or a second aliquot of said first sample, and contacting said second sample with ¹²C-PIC; thereby labeling at least one peptide with ¹²C-PIC in said second sample;
analyzing said second sample to validate ¹²C-PIC-labeling of at least one peptide;
comparing the results of the analysis of said first sample with the results of the analysis of said second sample, thereby identifying at least one peptide in a sample.

11. The method of claim 10, further comprising obtaining a third sample, wherein said third sample comprises and otherwise identical sample or a third aliquot of said first sample, and contacting said third sample with a third label; thereby labeling peptides in said third otherwise identical sample or said third aliquot;
analyzing said third sample to validate labeling of at least one peptide with said third label;
comparing the results of the analysis of the peptides labeled with ¹³C-PIC with the results of the analysis of the peptides labeled with ¹²C-PIC, with the results of the analysis of the peptides labeled with the third label, thereby identifying peptides in a sample.

12-14. (canceled)

15. The method of claim 10, wherein said method identifies the charge of a peptide ion labeled with ¹³C-PIC or ¹²C-PIC.

16. The method of claim 10, wherein said first sample and said second samples are combined following labeling, and then said combined sample is analyzed.

17. The method of claim 10, wherein said comparison of results comprises manual algorithms or computer assisted algorithms to compare peptides.

18-20. (canceled)

21. The method of claim 10, wherein said samples are analyzed using LC/MS, further wherein spectra are identified using a database search algorithm.

22. The method of claim 21, wherein said database search algorithm is selected from the group consisting of SEQUEST, MASSCOT, and OMSSA.

23-24. (canceled)

25. The method of claim 10, wherein b-ions and y-ions are identified using MS2 fragmentation.

26. The method of claim 10, wherein said first and second samples are selected from the group consisting of normal tissue samples, diseased tissue samples, biopsies, cultured cells, blood, saliva, feces, cerebrospinal fluid, semen, tears, and urine.

27-28. (canceled)

29. The method of claim 10, wherein said peptides are quantified by a data collection procedure using LC/MS/MS, said procedure comprising first acquiring a wide range MS1 spectrum, then collecting paired sets of spectra, wherein said paired sets of spectra comprise a narrow range 15 m/z MS1 scan of a selected ion followed by an MS2 scan of the same target ion.

30. The method of claim 10, further wherein MS2 fragmentation identifies amino-terminal amino acids.

31-33. (canceled)

34. A method for identifying at least one biomarker associated with a disease or disorder, said method comprising;
obtaining a first sample from a subject with said disease or disorder and analyzing said sample according to the method of claim 10,
obtaining a second otherwise identical sample from an unaffected subject and analyzing said second sample according to the method of claim 10;
comparing the results of the analysis of the first sample with the results of the analysis of the second sample;
wherein a higher or lower level of a peptide in said first sample compared to the level of said peptide in said second otherwise identical sample, is an indication that said peptide is a biomarker of said disease or disorder, thereby identifying a biomarker associated with a disease or disorder.

35. The method of claim 34, wherein said disease is cancer.

36. The method of claim 35, wherein said cancer is breast cancer.

37. The method of claim 34, wherein said first sample is selected from the group consisting of diseased tissue samples, biopsies, cultured cells, blood, saliva, feces, cerebrospinal fluid, semen, tears, and urine.

38. A method for diagnosing a disease or disorder associated with said biomarker of claim 34, said method comprising obtaining a sample from a test subject, comparing the level of said biomarker in said test subject with the level of said biomarker from an otherwise identical sample from an unaffected subject or from an otherwise identical unaffected sample from said test subject, wherein a higher or lower level of said biomarker in said sample from a test subject, compared with the level of said biomarker in said sample from an unaffected subject or from an unaffected sample from said test subject, is an indication that said test subject has said disease or disorder associated with said biomarker.

39. The method of claim 38, wherein said first sample is selected from the group consisting of diseased tissue samples, biopsies, cultured cells, blood, saliva, feces, cerebrospinal fluid, semen, tears, and urine.

40. The method of claim 38, wherein said disease is cancer.

41. The method of claim 40, wherein said cancer is breast cancer.
